(19) United States
(12) Patent Application Publication
(10) Pub. No.: US 2004/0219570 A1
(43) Pub. Date: Nov. 4, 2004

(54) Methods of directed evolution

(76) Inventor: John C. Salerno, Averill Park, NY (US)

Correspondence Address: ELMORE CRAIG, P.C., 209 MAIN STREET, N. CHELMSFORD, MA, 01863, US

(21) Appl. No.: 10/712,806

(22) Filed: Nov. 13, 2003

Related U.S. Application Data
(60) Provisional application No. 60/446,045, filed on Feb. 6, 2003.

(51) Int. Cl. C12Q 1/68; C12P 19/34

(52) U.S. Cl. 435/6; 435/91.2

(57) ABSTRACT

Methods for generating chimeric polynucleotides by directed evolution are described. In the methods, splice points of interest are identified within the polynucleotides of a basis set of polynucleotides, preferably through the use of an algorithm that defines the number of splice points and selects the splice points, either by random selection or using information regarding alignment of the polynucleotides. The algorithms can include additional factors, including a definition of a desired distance between splice points, and/or weighing factors to bias selection of splice points. Chimeric polynucleotides are generated using primers (e.g., double primers or non-overlapping primers) and polymerase chain reaction or combinatorial strategies.

### RELATED APPLICATION(S)

This application claims the benefit of U.S. Provisional Application No. 60/446,045, filed on Feb. 6, 2003. The entire teachings of the above application(s) are incorporated herein by reference.

### BACKGROUND OF THE INVENTION

Production of proteins with novel properties has been a goal of the biotechnology industry and the basic life science research community for several decades. Proteins to be engineered include enzymes (engineered for novel chemistries, substrate specificities, altered solubility or altered stability); receptors; antibodies (engineered for altered ligand recognition); DNA binding proteins (engineered to recognize new sites or to provide signals of events inside the cell); and other proteins. Two major paths to the desired end are rational design and directed evolution.

One type of rational design includes de novo approaches in which a sequence not directly related to existing protein is specified and synthesized to produce a folded entity. The knowledge of protein folding, however, is insufficient for the practical production of novel proteins. Another approach for rational design uses existing proteins and incorporating specific alterations (e.g., modifications of amino acid residues to alter substrate or cofactor specificity). For example, a successful though limited approach is the production of fusion proteins in which two or more genes are combined in frame to produce a protein in which the regions coded for by the parent genes independently fold but are joined by a linking region.

The introduction of directed evolution methods to the problems of protein and pathway design has attracted considerable attention and excitement in the last decade. While rational protein design has made progress, the idea of using a method based on natural selection to develop new enzymes and structures has great appeal. Initial methods of directed evolution were based on cycles of mutagenesis and selection (see, e.g., Shao, Z. and Arnold, F. H., Curr. Opin. Struct. Biol. 6(4):513-8 (1996)).

Although successes were recorded using this strategy, many attempts to evolve enzymes with desired characteristics were failures for reasons which were not always well understood. Furthermore, in directed evolution, as in natural selection, a pathway from the starting material to the desired resultant material must exist in which all the intermediates are reasonably successful. A weakness in this procedure is the need to proceed in very small jumps, restricting the volume of evolutionary space that is accessible.

More recently, methods loosely termed “gene shuffling” have been attempted (see, e.g., Crameri, A., et al., Nature 391(6664):288-291 (1998)). Initially, a basis set of homologous genes was restricted and the fragments randomly ligated. Most of products in such a protocol were nonsense DNA, but in a small minority of the cases, homologous fragments of related genes were ligated in the correct order. By applying selection criteria to a host transformed with the mixed DNA, a relatively small number of chimeras with desirable new features could be identified. A chimeric gene (or gene product) contains regions derived from two or more parent genes; to have a reasonable chance of stable folding, chimeric proteins were derived from genes composed of fragments from a basis set of related genes combined in frame and in order. This method allowed production of stably folded chimera which differ from the basis genes by more than a few point mutations, and provided additional evolutionary pathways that were not generally accessible by natural evolution. However, only a very small percentage of fragments were produced which had the potential to fold stably and have the desired activity. Furthermore, the number of potential chimeras which make up a region of evolutionary space spanned by a basis set are enormous.

Introduction of the polymerase chain reaction (PCR) into methods of directed evolution (see, e.g, Crameri, A., et al., Nature 391(6664):288-291 (1998); Newton, C. R. and Graham, A., PCR (BIOSis Scientific Publishers, Oxford, U.K., 1994); and Pelletier, J. N., Nat. Biotechnol. 19(4):314-5 (2001)) allowed ordered connection of related DNA fragments at natural splice sites. However, because fragments must prime each other with reasonable melting and annealing temperatures, splices between two genes occur only in regions of high similarity, as they require sufficient relatedness to allow mutual priming. Furthermore, methods for producing and screening all possible chimera are not yet known.

The present inventor has previously filed on improved methods of producing chimeras which include selecting a basis set of polynucleotides, identifying splice points within the basis set, generating double primer sets for each splice point and using the double primers in a polymerase chain reaction to amplify combinations of fragments. This method, and the algorithms used therein, utilized areas of homology between the polynucleotides to select “natural crossovers” which can be used to design double primers. However, a need remains for a method to sample evolutionary space in a productive way.

### SUMMARY OF THE INVENTION

The present invention is drawn to methods of generating chimeric polynucleotides, for purposes including directed evolution and other gene shuffling strategies. The invention includes a method for generating a library of chimeric polynucleotides, comprising:

a) aligning the sequences of a basis set of polynucleotides, wherein the basis set comprises three or more different polynucleotides;

b) identifying areas of homology between each polynucleotide in the basis set and at least one other polynucleotide in the basis;

c) identifying splice points in each polynucleotide in the basis set which correspond to areas of homology identified in step (b);

d) preparing a set of oligonucleotide double primers, wherein each double primer comprises a “pre” region joined to and followed immediately by a “post” region, and wherein the “pre” region comprises an oligonucleotide primer for a splice point in one polynucleotide identified in the basis set, and the “post” region comprises the complement of an oligonucleotide primer for the corresponding splice point in another polynucleotide in the basis set, and wherein two or three of the following are satisfied: (i) the set of double primers includes double primers comprising exact matches or near matches for all possible combinations of pre and post regions for each splice point; (ii) at least one double primer is capable of priming more than one polynucleotide in the basis set in the pre and/or post regions; and (iii) the set includes at least one double primer that does not prime at least one polynucleotide in the basis set;

e) hybridizing under hybridization conditions said double primers to said basis set or fragments thereof comprising said splice points, thereby generating hybridized complexes;

f) amplifying in a polymerase chain reaction the hybridized complexes of step (e), thereby generating a library of chimeric polynucleotides,

wherein each chimeric polynucleotide comprises a fragment from at least two of the polynucleotides in the basis set and fragments of each polynucleotide are incorporated into the library.

The invention also includes a method for generating a library of chimeric polynucleotides, comprising:

a) aligning the sequences of a basis set of polynucleotides, wherein the basis set comprises two or more different polynucleotides;

b) identifying areas of homology between each polynucleotide in the basis set;

c) identifying splice points in each polynucleotide in the basis set which correspond to areas of homology identified in step (b);

d) preparing a set of oligonucleotide double primers, wherein each double primer comprises a “pre” region joined to and followed immediately by a “post” region, and wherein the “pre” region comprises an oligonucleotide primer for a splice point in one polynucleotide identified in the basis set, and the “post” region comprises the complement of an oligonucleotide primer for the corresponding splice point in another polynucleotide in the basis set, and wherein the set of double primers includes double primers comprising all possible combinations of pre and post regions for each splice point;

e) preparing a set of blocking oligonucleotides that hybridize to regions of the basis polynucleotides;

f) hybridizing under hybridization conditions said double primers and blocking oligonucleotides to said basis set or fragments thereof comprising said splice points, thereby generating hybridized complexes;

g) amplifying in a polymerase chain reaction the hybridized complexes of step (e) under non strand displacing conditions, whereby the blocking oligonucleotides interrupt the reaction thereby generating a library of chimeric polynucleotides,

wherein each chimeric polynucleotide comprises a fragment from at least two of the polynucleotides in the basis set and fragments of each polynucleotide are incorporated into the library.

The methods can be used to generate chimeras between polynucleotides which have high or low homology (or regions of high and/or low homology) to one another, or combinations thereof. The basis set can include three, four, five, six, seven, eight, nine, ten or more polynucleotides. Polynucleotides having a high homology, as that term is used herein, include polynucleotides that include sequence regions, preferably each independently having 5, 6, 7, 8, 9, 10, or more nucleotides, each of which hybridizes to the reverse complement of the other with a Tₘ of at least 55° C., and preferably with a Tₘ of at least 60° C. Such regions can define natural splice ‘regions’ or points.

The present invention permits the introduction of flexibility in identifying splice points or cross-over junctions. Thus, upon analyzing the sequences of the basis set, it is possible that a desirable splice point or cross-over junction is located that doesn't permit good crossover between all polynucleotides within the basis set without use of a large number of double primers. Thus, subsequence A1 may permit generating good to excellent double primers that will hybridize to polynucleotides A, B, D, E, F and G but will not hybridize to C. The method of this invention includes the step of analyzing the sequences of the basis set and the sequence of C to identify, preferably adjacent sequences to A1 (and/or the homologous regions of B, D, E, F or G), for additional splice points that will create crossover junctions between C and one or more or all of the members of the basis set.

In one embodiment, the basis set comprises at least a first polynucleotide, a second polynucleotide and a third polynucleotide; said first and second polynucleotides have at least one area of high homology corresponding to a first splice point in said first and second polynucleotides thereby identifying a first splice point for priming a first double primer; and said third polynucleotide has an area of low homology with said first polynucleotide which corresponds to said first splice point whereby said first double primer does not prime said third polynucleotide under the conditions of steps (e) and (f).

In another embodiment, the invention comprises the step of identifying an area of high homology between said first and third polynucleotides in an area adjacent to said first splice point, thereby identifying a second splice point for priming with a second double primer said first and third polynucleotides. Thus, one polynucleotide can have multiple splice points within the same general region which will hybridize to multiple distinct double primers and create crossovers between polynucleotides within the basis set which would not be possible otherwise. This can result in a number of splice points in the first polynucleotide which is greater than the number of splice points in the second and/or third polynucleotides within the basis set. Thus, the invention can include a step of identifying an area of high homology between said second and third polynucleotides in an area adjacent to said first splice point, thereby identifying a third splice point for priming a third double primer between said second and third polynucleotides.

In one embodiment, the ends of the double primer can hybridize to multiple members of the basis set. For example, the “pre” region of at least one double primer hybridizes to at least two polynucleotides and/or the “post” region of at least one double primer hybridizes to at least two polynucleotides.

The invention can be practiced with full length polynucleotides or with fragments of the basis set or combinations of both. The polynucleotides can be natural or synthetic, genomic, cDNA or RNA or PNA and can comprise an entire gene, coding sequence of a gene or a fragment thereof.

The invention can conveniently be practiced employing an algorithm, such as can be programmed and executed by a computer processing unit. The algorithm can be designed to require input of a minimum distance between splice points, blocking oligonucleotides and/or number of double primers per polynucleotide, to bias selection of splice points between or incorporation of regions of interest in the polynucleotides or structural elements or motifs, or within regions of sequence homology or identity.

The invention can be designed to be conducted in vivo, in vitro (e.g., in solution) or in silico. Thus, in one embodiment, the method further comprises a step of contacting a chip characterized by a set of primers which hybridize to one or more of the terminal sequences of the polynucleotides in the basis set with the basis set, whereby the library of chimeric polynucleotides is generated on said chip.

In yet another embodiment of the invention, which can be used in combination with or in the alternative to other embodiments described herein, the method comprises contacting said polynucleotides of said basis set with one or more blocking oligonucleotides having high complementarity with at least one region of at least one of said polynucleotides, whereby each of said oligonucleotides hybridizes to said polynucleotide and interrupts the polymerase chain reaction. The blocking oligonucleotides can incorporate a covalent modification of the 3′ end or a non-complementary base at the 3′ end to prevent extension. In general, any modification preventing 3′ extension while allowing for hybridization to the target sequence is potentially suitable.

The methods comprise generation of a prespecified set of chimeric polynucleotides, which can be further facilitated by prior, or subsequent, gene shuffling. In the methods, a basis set of polynucleotides comprising three or more different polynucleotides is preferably used. One or more of the polynucleotides of the basis set can comprise whole genes; alternatively, none of the polynucleotides of the basis set can comprise whole genes. If desired, one or more of the polynucleotides of the basis set can include synthetic nucleic acids, and/or can incorporate one or more non-natural splice points.

Splice points of interest are identified within the polynucleotides of the basis set, wherein each polynucleotide in the basis set has the same number of splice points. This permits the maximum number of chimera which can be obtained from the basis set and/or for a given number of primers. The splice points can be identified by use of an algorithm that defines the position of naturally occurring splice points (defined by regions of homology sufficient to allow fragments to prime each other). For synthesis methods which do not depend on natural homology, splice points can be identified by random selection; alternatively, they can be identified using information regarding alignment of the polynucleotides. Algorithms can include additional factors, including a definition of a desired distance between splice points, and/or weighing factors to bias selection of splice points, such as weighing factors that bias selection of splice points in regions of interest in the polynucleotides of the basis set; that bias selection of splice points in regions having a preselected percentage of homology among the polynucleotides of the basis set; and/or bias selection of splice points in structurally identifiable regions of the polypeptides encoded by the polynucleotides of the basis set.

It is possible to bias splice point selection, and primer selection, and blocking oligonucleotide selection to require that specific segments from a basis gene are incorporated, and others excluded. Inclusion of natural splice site information can be used at this stage to bias splice point selection to reduce the number of double primers required.

In one embodiment, double primers are used to generate the chimeric polynucleotides. Oligonucleotide double primer sets are created for each splice point, in which each double primer in a set comprises a “pre” region joined to and followed immediately by a “post” region. The “pre” region comprises an oligonucleotide primer for a splice point in one polynucleotide in the basis set, and the “post” region comprises the complement of an oligonucleotide primer for the corresponding splice point in another polynucleotide in the basis set. The set of double primers includes double primers comprising all possible combinations of pre and post regions for each splice point. The double primer sets are used in the polymerase chain reaction to amplify combinations of fragments, thus generating a multitude of chimeric polynucleotides, in which each chimeric polynucleotide comprises a fragment from at least two of the polynucleotides in the basis set.

Additional steps can be included to limit production of certain chimeric polynucleotides in favor of other chimeric polynucleotides: for example, one or more “polishing” steps can be included during polymerase chain reaction, in which loose single stranded ends of products are briefly digested with an exonuclease. In another example, one or more “poisoned primers” can be used, where the poisoned primers hybridize with high stringency to a product which is incapable of supporting polymerase chain reaction, thereby interrupting extension during polymerase chain reaction.

The methods describe herein allow flexible generation of novel chimeric polynucleotides, from which polypeptides can be prepared. The methods provide a productive sample of evolutionary space for the polynucleotides in the basis set, and allow use of polynucleotides in the basis set that are not closely homologous, thereby producing chimeric polynucleotides previously unavailable by traditional modes of directed evolution.

### DETAILED DESCRIPTION OF THE INVENTION

The present invention pertains to methods for generating chimeric polynucleotides, such as polynucleotides encoding polypeptides (“chimeric polypeptides”), using directed evolution of a basis set of polynucleotides. The inventions are particularly suited for application to the method described in WO02/090496, published Nov. 14, 2003 and corresponding U.S. Ser. No. 10/138,183 filed on May 2, 2003, which are incorporated herein by reference.

Basis Set of Polynucleotides

As described herein, a “polynucleotide” is a polymeric chain of nucleotides (e.g., a gene, gene fragment, cDNA, mRNA), and a “polypeptide” is a polymeric chain of amino acids (e.g., a protein). A “basis set” is a group of 2 or more polynucleotides, preferably greater than or equal to 3 polynucleotides, such as between 3 and 12 polynucleotides, inclusive, or more; the basis set of polynucleotides is used as the starting materials for the directed evolution. The polynucleotides of the basis set can be of any length; generally, they are greater than 20 nucleotides in length (e.g., approximately 50 nucleotides in length or greater, preferably approximately 75 nucleotides in length or greater, more preferably approximately 100 nucleic acids in length or greater, more preferably approximately 500 nucleic acids in length or greater); if desired, only a short fragment of any one of the polynucleotides is used during generation of chimeric polynucleotides. In one embodiment, the basis set comprises at least two polynucleotides that have a high degree of sequence homology or identity; in a preferred embodiment, at least two of the polynucleotides of the basis set have sufficient homology to one another to anneal for priming during polymerase chain reaction. In another embodiment, the basis set comprises at least two polynucleotides that encode polypeptides having structural homology in one or more regions.

To determine the percent homology or identity of two nucleic acid sequences, the sequences are aligned for optimal comparison purposes (e.g., gaps can be introduced in the sequence of one nucleic acid molecule for optimal alignment with the other nucleic acid molecule). The nucleotides at corresponding nucleotide positions are then compared. When a position in one sequence is occupied by the same nucleotide as the corresponding position in the other sequence, then the molecules are homologous at that position. As used herein, nucleic acid “homology” is equivalent to nucleic acid “identity”. The percent homology between the two sequences is a function of the number of identical positions shared by the sequences (i.e., percent homology equals the number of identical positions/total number of positions times 100). In preferred embodiments, at least two polynucleotides in the basis set have at least 50% homology or greater; more preferably, 70% homology or greater; even more preferably, 80% homology or greater; still more preferably, 90% homology or greater. “High” homology, as used herein, refers to 80% homology or greater.

In one embodiment of the invention, one or more of the polynucleotides of the basis set comprise full length genes. A “gene,” as used herein, refers to a specific sequence of nucleotides (e.g., DNA or RNA), typically locatable on a chromosome, that encodes a particular polypeptide (e.g., a protein). In another embodiment of the invention, one or more of the polynucleotides of the basis set comprise partial genes (for example, a polynucleotide comprising one or more exons of a gene). In still another embodiment of the invention, the polynucleotides of the basis set comprise synthetic nucleotide sequences.

The polynucleotides of the basis set can include naturally-occurring nucleic acids (e.g., nucleic acids that are found in an organism, for example, genomic DNA, complementary DNA (cDNA), chromosomal DNA, plasmid DNA, mRNA, tRNA, and/or rRNA). The polynucleotides can also comprise modified nucleic acids. “Modified” nucleic acids include, for example, nucleic acids which are naturally-occurring, as described above, but are modified to alter (e.g., add, delete, or modify) one or more nucleotides. In another embodiment, the polynucleotides of the basis set can include synthetic nucleic acids, including but not limited to, nucleic acids prepared on solid phases using well-known and/or commercially-available procedures, e.g., using an automated nucleic acid synthesizer. In yet another embodiment, a combination of more than one type of nucleic acid can be present (e.g., naturally-occurring and/or modified and/or synthetic nucleic acids). If desired, the naturally-occurring, modified and/or synthetic nucleic acids can comprise modified nucleotides. As used herein, a modified nucleotide is a nucleotide that has been structurally altered so that it differs from a naturally-occurring nucleotide.

The polynucleotides of the basis set can be obtained from various biological and/or chemical materials using standard procedures. For example, naturally-occurring polynucleotides (e.g., genes) can be obtained from organisms, tissues, and/or cells from veterinary or human clinical test samples collected for diagnostic and/or prognostic purposes. For example, cells can be lysed and the resulting lysate can be processed using techniques familiar to one of skill in the art to obtain an aqueous solution of nucleic acid (e.g., DNA and/or RNA) (see, for example, Ausebel, F., et al., Current Protocols in Molecular Biology, Wiley, New York (1988); Maniatis, et al., Molecular Cloning: A Laboratory Manual, Cold Spring Harbor Laboratory, Cold Spring Harbor, N.Y. (1982)). Nucleic acids, where appropriate can also be cleaved to obtain a fragment that contains a desired polynucleotide, for example, by treatment with a restriction endonuclease or other site-specific chemical cleavage methods. Polynucleotides can also be synthesized from nucleotide monomers, e.g., using an automated nucleic acid synthesizer, or can be obtained using recombinant DNA methodology.

If desired, the polynucleotides of the basis set can be modified by introducing features that will facilitate directed evolution. For example, common restriction sites recognized by particular enzymes can be introduced into a polynucleotide by standard techniques (e.g., site directed mutagenesis, such as by PCR-based mutation). An “introduced” or “non-native” restriction site, as used herein, is a restriction site that is incorporated into a polynucleotide at a point where a restriction site was not previously present, or at a point where the alignment had natural homology insufficient for cross-sequence priming. For example, a different restriction site (e.g., a restriction site recognized by a different enzyme) was previously present can be incorporated. In a preferred embodiment, the restriction sites can be introduced without affecting the amino acid sequence encoded by the polynucleotide, due to the degeneracy of the code. A common restriction site can be, for example, a short region suitable for priming, such as a designated splice position from one sequence which is used to replace its cognates in all the other polynucleotides in the basis set.

Design of Chimeric Polynucleotides: “In Silico” Preparation

In the methods of the invention, chimeric polynucleotides are designed, based on the polynucleotides of the basis set. A “chimeric polynucleotide,” as used herein, is a polynucleotide that contains fragments from at least two of the polynucleotides in the basis set. In a preferred embodiment, the chimeric polynucleotide contains one or more fragments from each polynucleotide in the basis set. A “fragment” of a polynucleotide, as used herein, is less than the whole polynucleotide: for example, if a polynucleotide in the basis set is 300 nucleotides in length, a fragment of that polynucleotide comprises from 1 to 299 consecutive nucleotides of the polynucleotide. Usually, the fragment will contain that part of the polynucleotide that is between two splice points in the polynucleotide, or that part of the polynucleotide that is between an end (i.e., a 5′ or 3′ end) of the polynucleotide and a splice point in the polynucleotide. A “splice point” in a polynucleotide is the location at which the polynucleotide is fragmented.

To generate chimeric polynucleotides, splice points of interest within the polynucleotides of the basis set are identified. Each polynucleotide in the set will have the same number of splice points in silico, although not all of the fragments between splice points need be used when generating chimeric polynucleotides in vitro. In one embodiment, an algorithm which defines and aligns natural splice points within the polynucleotides of the basis set is used. In another embodiment, an algorithm that can select one or more random splice points, is used. As used herein, the term “algorithm” refers to step-by-step procedure for solving a problem (e.g., the identification of splice points) in a finite number of steps that frequently involves repetition of an operation, preferably (though not necessarily) with the assistance of computer.

In either embodiment, the algorithm can incorporate desired parameters, including: the number of splice points desired and alignment of the sequences in the basis set. In additional embodiments, the algorithm can further include parameters relating to a desired distance between splice points (e.g., approximately 8-20 base pairs apart, to facilitate PCR priming); if desired, the algorithm can additionally include parameters relating to melting temperatures of hybridized fragments of the polynucleotides of the basis set (e.g., Tmax and Tmin; for example, a Tm between about 50-75° C., inclusive).

If desired, a preliminary step can be added in which splice points are identified which lie in regions of interest in the polynucleotide sequences of the basis set (e.g., regions in which the homology is favorable for hybridization during polymerase chain reaction, to allow for reduced numbers of double primers (PCR)). A pairwise sliding box investigation of the number of exact matches can be formed; this will be quicker than the calculation using Tm, because no floating point calculations are needed. Sequence regions predicted to be of low utility could be discarded from the areas used for splice points, and sequences of low utility within a specified fragment could also be discarded. Splice points within the homologous regions could then be identified without searching the entire alignment. This preliminary step is particularly useful for constructing chimera using PCR (as described below), for example, when the basis set comprises a set of overlapped oligonucleotides taken from a superfamily alignment; some sequences might contribute only one oligonucleotide, corresponding to a short fragment of a polynucleotide, to the set of chimera.

Alternatively or in addition, if desired, the algorithm can incorporate “weighing” or “biasing” factors. In one embodiment, favorable regions for splice points can be identified using a specified region or a specified number or exact matches in a specified region as a cutoff criterion. For example, the biasing factors can be set so that specific splice points (such as those near the beginning or the end of the polynucleotide) can be rejected. Sets of splice points within specified regions can be identified from Tm calculations, and other sequences added to the natural sets by incremental adjustment of each polynucleotide in the basis set until Tmin is reached with the consensus sequence of the natural set. In one embodiment, the Tm is set to be approximately 50-75° C., inclusive; this will typically correspond to hybridizing 14-20 base pair regions with about 2 mismatches. The weighing factors can be designed to bias the selection of splice points in regions of the polynucleotides of the basis set that have particular homology (e.g., high homology, or low homology); alternatively or in addition, the weighing factors can incorporate structural “mask” for selection of splice points, which will bias the selection of splice points in structurally identifiable regions of the polypeptides encoded by the polynucleotides of the basis set (e.g., intervening regions; loops; transmembrane sequences; domain or subdomain boundaries; borders and internal divisions of binding sites for cofactors, ligands, prosthetic groups; and borders and internal divisions for control elements, etc.).

For example, in one embodiment of an algorithm, starting with the polynucleotides of the basis set, a sequence alignment in array form A(i,j), where i is the number of the polynucleotide and j the sequence position in the alignment; A(i,j) can be a base character or a blank. A sliding box algorithm brings a box of width n down the alignment, calculating the melting temperature T (i,j) for all base pairs at each position. This calculation can include mismatches, if desired. If a majority of the T(i,j) is high, n is decreased and the T(i,j) are recalculated until the maximum number are between specified limits of Thot and Tcold. The number of T(i,j)s within the limit is stored, along with the initiation point and the box size. The best m overlaps can be reported. This method works particularly well for basis sets having highly homologous sequences.

In another embodiment of an algorithm, the algorithm calculates all the pairwise values for Tmax and Tmin for T(n,m,k,l) between sequences n and m for fragments beginning at position k and extending l bases. Every T(n,m,k,l) between Thot and Tcold generates a pair a(i,j,n) and a(i′,j′,m) corresponding to the fragments in sequences n and m for which it was calculated.

Every a(i,j,n) can be represented as an element A1, and a table can be constructed using the pairs. For example, the element A1 hybridizes with the desired melting temperature to B1, B2, C1, D1, and D2 (all the A elements would have the same n value, and for each table all the A elements would start at the same position but be of different lengths). In addition, B1 hybridizes as desired with C1, C2 and D2, and so on. A fully connected set of elements Aw, Bx, Cy and Dz is generated such that Bx, Cy and Dz all appear under Ay, Cy and Dz appear under Bx, and Dz appears under Cy.

This method can be performed using by a tree algorithm in which each branch originating in column A1 is followed to completion. For example, B1 can be followed to C1, which is also found in A1. C1 is followed to D1, which is found in A1 but not in B1. The missing element in B1 generates a penalty of 1 for this branch. The next branch to be investigated extends from A1 to B1 to C1 to D2, which is found in A1 and B1 as well. There is no penalty, since the fragments represented by the elements can span the sequence set at this position. There will not always be such a set of elements at an arbitrary position, so the set of elements, and hence fragments, with the lowest penalty at each position is recorded along with its penalty score. An arbitrary number of “best” splice points can be reported. If no zero or single penalty sets are identified for a particular sequence position, a second table can be constructed starting with the B elements to identify potential sets missing only the A element, etc., until a specified cutoff is reached. In one embodiment, a preliminary step as described above, in which splice points are identified which lie in regions of interest in the polynucleotide sequences of the basis set, e.g., regions in which the homology is favorable for hybridization during polymerase chain reaction (PCR), can be added to this algorithm.

In a third embodiment of the algorithm, a heuristic algorithm can be used for the identification of overlapping oligonucleotide sets in the basis set of polynucleotides, in order to prepare chimeric polynucleotides as described in detail below. This algorithm begins by identifying favorable regions in an alignment using the number of exact matches in a specified region as the cutoff criterion, as in the preliminary step described above. ‘Natural’ sets within these regions are identified from Tm calculations, and other sequences are added to the natural sets by incremental adjustment of each sequence to be added until T_{low} is reached with the consensus sequence of the natural set. For example, one sequence can be assigned as the master sequence at each splice point; this can be done by arbitrary assignment, or by choosing the sequence with the best local overlap with other members of the set. Sequences with low annealing temperatures can be forced to anneal by progressively substituting codons from the master sequence for mismatched codons. This minimal approach preserves maximum diversity at splice points; in extreme cases complete substitution at a splice point can be used to force annealing between previously unrelated oligonucleotides. This algorithm is particularly useful for basis sets of polynucleotides having low homology with one another, as it assists in the construction of a set of overlapped oligos in which the original gene sequences have been modified to produce favorable overlaps for polymerase chain reaction (PCR). The chimeric polynucleotides prepared by these methods have a much higher diversity than would be produced by random breakage or restriction, since overlaps among the polynucleotides of the basis set are optimized.

Often, a splice point (“a consensus splice point”) identified for some of the polynucleotides in the basis set are not suitable for one or more polynucleotides in the basis set. Once this has been identified, the algorithm then compares the sequences of the polynucleotides to identify one or more additional splice points (“a non-consensus splice point”) for the remaining polynucleotide(s) to complete the set. Often, the additional splice point will be located proximal or nearly proximal to the corresponding consensus splice point(s) for the other polynucleotides. For example, the additional, non-consensus splice point can be within 300, 200, 100, 50, or 30 base pairs of the corresponding consensus splice point.

Once splice points are identified, chimeric polynucleotides can be generated using a variety of methods presented below. Representative algorithms for identifying splice points are described. In certain embodiments, polymerase chain reaction-based synthesis using double primers, can be used.

Preparation of Chimeric Polynucleotides: Splice Point Selection

In one embodiment of the invention, a sequence alignment A(i,j) as described above uses a set of homologous polynucleotides as the basis set for chimera formation. In the most basic variant, the number of splice points desired is specified, and the splice points are chosen by repeated random selection without replacement. The basic selection mechanism is the use of a random number generator to yield a position in amino acid space, followed by multiplication by three to convert to nucleotide space at codon boundaries (as described in detail below). An alignment A(i,j) where i is the sequence designator and j the position is used. For a set of ordered splice points M(h) the chimeric sequences are generated by combinatorial concatenation so that to each vector component Pre(i,j) (j=1,M(1)−1) I vectors are formed by adding the strings A(i,j) (i=M(1),M(2)−1). All the available components are concatenated with all the existing vectors at each splice. For example, starting with the I strings Pre(i,j), a set of 10 sequences would have 10 pre components, 100 chimera after the first splice, etc., forming 10⁶ vectors after five splices. Splice points can generally be constrained to be a sufficient length apart (e.g., at least 12-20 bp) apart to allow for PCR priming; this can be done by discarding random selections which do meet the specified criteria. Alternatively, splice points closer than this can be allowed but treated differently than well spaced splices.

Information from the investigation of natural splice points described previously can be incorporated during the biasing to reduce the number of double primers which would be required for polymerase based synthesis.

Preparation of Chimeric Polynucleotides I: Polymerase Chain Reaction (PCR)-Based Methods using Double Primers

In another embodiment of the invention, generation of polynucleotide chimera is conducted by preparation of oligonucleotide “double primers” based on splice points. “Primers” are oligonucleotides that hybridize in a base-specific manner to a complementary strand of nucleic acid molecules. Such probes and primers include polypeptide nucleic acids, as described in Nielsen et al., Science, 254, 1497-1500 (1991). In a preferred embodiment, a “primer” refers in particular to a single-stranded oligonucleotide which acts as a point of initiation of template-directed DNA synthesis using well-known methods (e.g., PCR, LCR) including, but not limited to those described herein. In a representative algorithm for double primer methods, the starting point of the algorithm is the alignment A(i,j) as previously described. It is not necessary to give the sequences of all the chimeric products to describe the primers, nor is it always desirable to do so because of the very large number of chimera which can be. In addition to A(i,j) i=1,I and j=1,J the number of splice points H and any biasing information which is desired, is included in the algorithm.

Each double primer in a double primer set comprises two regions (a “pre” and a “post” region): an oligonucleotide primer region for a polynucleotide in the basis set (“pre” region), joined to and followed immediately by an oligonucleotide primer region for the complement of that splice point for another polynucleotide in the basis set (“post” region). It is also possible to reverse the pattern by using double primers in which the post regions are primers for the post region and the pre regions are the complement of the primers for the pre regions of the strand. In any event, the double primers are intended to support an extension reaction in both directions on the appropriate strand. The double primers at each splice point M(h) are formed by the combinatorial concatenation of the pre and post subsequences. Better matches can be obtained by calculating the Tm for each pre and post with its complement and adjusting them by stepwise lengthening or shortening until the closest value to a desired Tm can be obtained for annealing of the entire primer to each gene. Gap characters in A(i,j) can be skipped so that pre and post are M characters in length before Tm adjustment.

Variations on this method can include biasing the selection to make the splice points more evenly spaced, or to make it probable that they be located in regions of high or low homology. Splice points can be concentrated in selected regions (e.g., loop regions or, conversely, regions of conserved secondary structure) or forbidden to lie in other regions, or a region in one of the sequences could be specified as an obligatory component of all of the chimera. In an extreme case, most of the chimera sequences can be constrained to be derived from a single polynucleotide in the basis set, and short elements can be swapped in at selected positions from other (e.g., homologous) polynucleotides in the basis set. Biasing can be performed at the level of checking for overlapped splices.

Overlapped splice regions can be discarded or given an alternative treatment because of hybridization possibilities between subsequences designed to prime basis set sequences and chimeric regions not present in the basis set. The most economical approach, other than the discard option, treats new splices with overlapped primer regions as alternative versions of the previous overlapped splice; a chimeric sequence could include a primer from the splice 2 set or the splice 2a set, but not both.

Using the methods described above, a set of splice points is defined in the polynucleotides of the basis set. For each splice point, an oligonucleotide double primer set is generated, so that the set of double primers includes double primers comprising all possible combinations of pre and post regions for each splice point. Using simple forward and reverse primers for each polynucleotide in the basis set, and a set of double primers for each splice point, a full set of chimera can be generated using polymerase chain reaction techniques. Polymerase chain reaction techniques are well known in the art (see, e.g., U.S. Pat. Nos. 4,683,202, 4,683,195, 4,965,188, and 4,683,202). The entire teachings of these patents are incorporated by reference herein.

Modifications to the Methods of Preparing Chimeric Polynucleotides

If desired, a solid phase can be used for attachment of the components during synthesis of the chimeric polynucleotides. The solid phase can be a solid medium, such as a microtiter plate, a membrane (e.g., nitrocellulose), a bead, a dipstick, a thin-layer chromatographic plate, a pin, a chip, or other solid medium.

Alternatively or in addition, if desired, unwanted PCR intermediates can be eliminated during synthesis of the chimeric polynucleotides, through the use of “poisoned primers”. A “poisoned primer” is a primer (nucleic acid) which hybridizes with high stringency to an intermediate which is incapable of supporting PCR, thereby interrupting extension between a viable forward primer and a viable reverse primer. For example, a modification of the 3′ end of a primer which prevents hybridization (e.g., addition of a non-homologous tail such as polyA) can be used. A small number of poisoned primers can often remove a large number of sequences from the pool of polynucleotides available for PCR.

The chimeric polynucleotides can be separated and characterized using standard techniques. For example, in one embodiment, MALDI-TOF mass spectroscopy can be used. MALDI-TOF MS allows biological polymers to be studies intact, and can provide accurate mass resolution to characterize the chimera distribution produced herein (see, e.g., Ross, P. L. et al., Anal. Chem. 70(10):2067-73 (1998)).

Production and Selection of Desired Polynucleotides

The chimeric polynucleotides can then be expressed, using standard techniques. For example, the chimeric polynucleotides can be introduced into a host cell for expression (see, e.g., Huse, W. D. et al., Science 246: 1275 (1989); Viera, J. et al., Meth. Enzymol. 153: 3 (1987)). The chimeric polynucleotides can be expressed, for example, in an E. coli expression system (see, e.g., Pluckthun, A. and Skerra, A., Meth. Enzymol. 178:476-515 (1989); Skerra, A. et al., Biotechnology 9:23-278 (1991)). They can be expressed for secretion in the medium and/or in the cytoplasm of bacteria (see, e.g., Better, M. and Horwitz, A., Meth. Enzymol. 178:476 (1989)); alternatively, they can be expressed in other organisms such as yeast or mammalian cells (e.g., myeloma or hybridoma cells). One of ordinary skill in the art will understand that numerous expression methods can be employed to produce chimeric polypeptides, encoded by the chimeric polynucleotides described herein. By fusing the chimeric polynucleotides to additional genetic elements, such as promoters, terminators, and other suitable sequences that facilitate transcription and translation, expression in vitro (ribosome display) can be achieved. Similarly, Phage display, bacterial expression, baculovirus-infected insect cells, fungi (yeast), plant and mammalian cell expression can be obtained.

Selection of chimeric polypeptides of interest can subsequently be performed by conducting assays to identify those chimeric polypeptides having a desired activity or function. The chimeric polypeptides can be screened by appropriate means for particular polypeptides having specific characteristics. For example, catalytic activity can be ascertained by suitable assays for substrate conversion and binding activity can be evaluated by standard immunoassay and/or affinity chromatography. Assays for these activities can be designed in which a cell requires the desired activity for growth. For example, in screening for polypeptides that have a particular activity, such as the ability to degrade toxic compounds, the incorporation of lethal levels of the toxic compound into nutrient plates would permit the growth only of cells expressing an activity which degrades the toxic compound (Wasserfallen, A., Rekik, M., and Harayama, S., Biotechnology 9: 296-298 (1991)). Chimeric polypeptides can also be screened for other activities, such as for an ability to target or destroy pathogens. Assays for these activities can be designed in which the pathogen of interest is exposed to the chimeric polypeptides, and those polypeptides demonstrating the desired property (e.g., killing of the pathogen) can be selected.

While this invention has been particularly shown and described with references to preferred embodiments thereof, it will be understood by those skilled in the art that various changes in form and details may be made therein without departing from the spirit and scope of the invention as defined by the appended claims.

What is claimed is:

1. A method for generating a library of chimeric polynucleotides, comprising:
a) aligning the sequences of a basis set of polynucleotides, wherein the basis set comprises three or more different polynucleotides;
b) identifying areas of homology between each polynucleotide in the basis set and at least one other polynucleotide in the basis;
c) identifying splice points in each polynucleotide in the basis set which correspond to areas of homology identified in step (b);
d) preparing a set of oligonucleotide double primers, wherein each double primer comprises a “pre” region joined to and followed immediately by a “post” region, and wherein the “pre” region comprises an oligonucleotide primer for a splice point in one polynucleotide identified in the basis set, and the “post” region comprises the complement of an oligonucleotide primer for the corresponding splice point in another polynucleotide in the basis set, and wherein two or three of the following are satisfied: (i) the set of double primers includes double primers comprising exact matches or near matches for all possible combinations of pre and post regions for each splice point; (ii) at least one double primer is capable of priming more than one polynucleotide in the basis set in the pre and/or post regions; and (iii) the set includes at least one double primer that does not prime at least one polynucleotide in the basis set;
e) hybridizing under hybridization conditions said double primers to said basis set or fragments thereof comprising said splice points, thereby generating hybridized complexes;
f) amplifying in a polymerase chain reaction the hybridized complexes of step (e), thereby generating a library of chimeric polynucleotides,
wherein each chimeric polynucleotide comprises a fragment from at least two of the polynucleotides in the basis set and fragments of each polynucleotide are incorporated into the library.

2. The method of claim 1 wherein at least two of the polynucleotides of the basis set have high homology to one another.

3. The method of claim 1 wherein at least two of the polynucleotides of the basis set have low homology to one another.

4. The method of claim 1, wherein the basis set comprises at least a first polynucleotide, a second polynucleotide and a third polynucleotide; said first and second polynucleotides have at least one area of high homology corresponding to a first splice point in said first and second polynucleotides thereby identifying a first splice point for priming a first double primer; and said third polynucleotide has an area of low homology with said first polynucleotide which corresponds to said first splice point whereby said first double primer does not prime said third polynucleotide under the conditions of steps (e) and (f).

5. The method of claim 4 further comprising the step of identifying an area of high homology between said first and third polynucleotides in an area adjacent to said first splice point, thereby identifying a second splice point for priming a second double primer between said first and third polynucleotides.

6. The method of claim 5 wherein the number of splice points in said first polynucleotide is greater than the number of splice points in said second and/or third polynucleotides.

7. The method of claim 5 further comprising the step of identifying an area of high homology between said second and third polynucleotides in an area adjacent to said first splice point, thereby identifying a third splice point for priming a third double primer between said second and third polynucleotides.

8. The method of claim 5 wherein the “pre” region of at least one double primer hybridizes to at least two polynucleotides.

9. The method of claim 8 wherein the “post” region of at least one double primer hybridizes to at least two polynucleotides.

10. The method of claim 1 at least one of the polynucleotides of the basis set comprises the entire coding sequence of a gene.

11. The method of claim 1 wherein at least one of the polynucleotides of the basis set comprises a synthetic nucleic acid.

12. The method of claim 1 wherein steps (a)-(c) are executed by use of an algorithm.

13. The method of claim 12, wherein the algorithm is executed by a computer processing unit.

14. The method of claim 13, wherein the algorithm requires input of a minimum distance between splice points and/or number of double primers per polynucleotide.

15. The method of claim 13, wherein the algorithm incorporates weighing factors to bias selection of splice points.

16. The method of claim 15, wherein the weighing factors bias selection of splice points between regions of interest in the polynucleotides of the basis set.

17. The method of claim 16, wherein the weighing factors bias selection of splice points in regions having a percentage of identity of at least about 90% between at least two of said polynucleotides in the basis set.

18. The method of claim 16, wherein the weighing factors bias selection of splice points between structurally identifiable regions of the polypeptides encoded by the polynucleotides of the basis set.

19. The method of claim 1 further comprising a step of contacting a chip characterized by a set of primers which hybridize to one or more of the terminal sequences of the polynucleotides in the basis set with the basis set, whereby the library of chimeric polynucleotides is generated on said chip.

21. The method of claim 1 further comprising contacting said polynucleotides of said basis set with one or more blocking oligonucleotides having high homology with at least one region of at least one of said polynucleotides, whereby each of said oligonucleotides hybridizes to said polynucleotide and interrupts the polymerase chain reaction of step (f).

22. The method of claim 21, wherein said blocking oligonucleotides are characterized by a covalent modification of the 3′ end.

23. The method of claim 21, wherein said blocking oligonucleotides are each characterized by a non-complementary base at the 3′ end.

24. A method for generating a library of chimeric polynucleotides, comprising:
a) aligning the sequences of a basis set of polynucleotides, wherein the basis set comprises two or more different polynucleotides;
b) identifying areas of homology between each polynucleotide in the basis set;
c) identifying splice points in each polynucleotide in the basis set which correspond to areas of homology identified in step (b);
d) preparing a set of oligonucleotide double primers, wherein each double primer comprises a “pre” region joined to and followed immediately by a “post” region, and wherein the “pre” region comprises an oligonucleotide primer for a splice point in one polynucleotide identified in the basis set, and the “post” region comprises the complement of an oligonucleotide primer for the corresponding splice point in another polynucleotide in the basis set, and wherein the set of double primers includes double primers comprising all possible combinations of pre and post regions for each splice point;
e) preparing a set of blocking oligonucleotides that hybridize to regions of the basis polynucleotides;
f) hybridizing under hybridization conditions said double primers and blocking oligonucleotides to said basis set or fragments thereof comprising said splice points, thereby generating hybridized complexes;
g) amplifying in a polymerase chain reaction the hybridized complexes of step (e) under non strand displacing conditions, whereby the blocking oligonucleotides interrupt the reaction thereby generating a library of chimeric polynucleotides,
wherein each chimeric polynucleotide comprises a fragment from at least two of the polynucleotides in the basis set and fragments of each polynucleotide are incorporated into the library.

25. The method of claim 24 further comprising the steps of (h) denaturing the product of step (g) and repeating steps (e) through (g), using the extension chimeric polynucleotides obtained from step (h) as primers in step (f).

26. The method of claim 25 wherein the blocking oligonucleotides correspond to a part of a double primer sequence.

27. The method of claim 25, wherein the algorithm requires input of at least one of the following: a minimum distance between splice points; number of double primers per polynucleotide; number of blocking oligonucleotides per polynucleotide; distance between a blocking oligonucleotide and an adjacent splice point.

28. The method of claim 27, wherein the algorithm incorporates weighing factors to bias selection of splice points and blocking oligonucleotides.

29. The method of claim 28, wherein the weighing factors bias selection of splice points and blocking oligonucleotides between regions of interest in the polynucleotides of the basis set.

30. The method of claim 24, wherein said blocking oligonucleotides are characterized by a covalent modification of the 3′ end.

31. The method of claim 24, wherein said blocking oligonucleotides are each characterized by a non-complementary base at the 3′ end.

32. A method for manufacturing chimeric polynucleotides, the improvement comprising the steps of (a) preparing a set of blocking oligonucleotides that hybridize to regions of the basis polynucleotides; (b) hybridizing under hybridization conditions said blocking oligonucleotides to a basis set of polynucleotides to be chimerized, thereby generating hybridized complexes; (c) amplifying in a polymerase chain reaction the hybridized complexes of step (b), whereby the blocking oligonucleotides interrupt the reaction thereby generating a library of chimeric polynucleotides.
